(19) United States
(12) Patent Application Publication
(10) Pub. No.: US 2015/0346092 A1
(43) Pub. Date: Dec. 3, 2015

(54) APPARATUS AND METHOD FOR MEASURING PROPERTIES OF FLUIDS

(71) Applicant: Electronics and Telecommunications Research Institute, Daejeon (KR)

(72) Inventors: Dae Sik LEE, Daejeon (KR); Moon Youn JUNG, Daejeon (KR); Myeong Soo LEE, Daejeon (KR); Wan Joong KIM, Daejeon (KR); Byoung Goo JEON, Daejeon (KR)

(21) Appl. No.: 14/691,086

(22) Filed: Apr. 20, 2015

(30) Foreign Application Priority Data
Jun. 3, 2014 (KR) 10-2014-0067592

(51) Int. Cl. G01N 21/59; G01N 33/49

(52) U.S. Cl. CPC G01N 21/59; G01N 33/49; G01N 2201/061; G01N 2201/068; G01N 2201/12

(57) ABSTRACT

Provided herein is an apparatus for measuring properties of a fluid, the apparatus including: a light emitting unit configured to emit a first light having a first wavelength and a second light having a second wavelength that is longer than the first wavelength, from outside a fluid accommodating unit where the fluid flows in and out to a measurement area inside the fluid accommodating unit; a light receiving unit disposed outside the fluid accommodating unit and configured to receive the first light and second light that passed the measurement area; and a measuring unit configured to measure the properties of the fluid based on an intensity of the first light and second light that the light emitting unit emitted and an intensity of the first light and second light that the light receiving unit received.

### CROSS-REFERENCE TO RELATED APPLICATION

The present application claims priority to Korean patent application number 10-2014-0067592, filed on Jun. 3, 2014, the entire disclosure of which is incorporated herein in its entirety by reference.

### BACKGROUND

1. Field of Invention

Various embodiments of the present disclosure relate to an apparatus and method for measuring fluids, and more particularly, to an apparatus and method for measuring a volume ratio of red blood cells to an whole blood.

2. Description of Related Art

A conventional blood analysis is made using large equipments, and is thus disadvantageous as it requires a time consuming preliminary operation, large amounts of specimen (blood), a long time for carrying the extracted specimen to an analyzing equipment, and a long time for analyzing the specimen if there are a large number of them to analyze. In order to overcome these disadvantages, there is a need for a small scale analyzing equipment that is capable of analyzing blood right after collecting the blood. By accommodating a small amount of blood in a biochip having a shallow channel (passage) and then putting the biochip into an apparatus that is capable of analyzing blood right away without a time consuming preliminary operation and then analyzing the blood, it is possible to overcome the aforementioned problems. To be used as a blood analyzing device in disease diagnosis in the related field, such an apparatus must have good reproducibility in the measurable concentration range, consume as small amount of power as to drive a battery, cost less in manufacturing, and be stable against environmental changes. Furthermore, it is necessary to develop s biochip analyzing apparatus and method capable of overcoming the problems that occur when there is only a small amount of specimen collected.

FIG. 1 is a view for explaining the problems in a conventional apparatus for measuring properties of a fluid. A conventional apparatus for measuring properties of a fluid is an apparatus for measuring a hematocrit accommodated in a biochip. A hematocrit is a volume ratio of red blood cells to an whole blood, which is important in diagnosing various diseases including anemia. In general, a low hematocrit indicates anemia, and a healthy male adult would show 42˜45% while a healthy female adult would show 38˜42% hematocrit. When using a conventional large scale analyzing apparatus, a large amount of blood is put into the apparatus, and then red blood cells are separated from the blood by a centrifuge, and then a volume of the whole blood is compared with a volume of the red blood cells.

In order to measure a hematocrit optically, an electromagnetic absorption ratio must be measured for at least to wavelengths. Referring to FIG. 1, a first light having a first wavelength is emitted to a first area (A1), and a second light having a second wavelength is emitted to a second area (A2), and then the electromagnetic absorption ratio for the first wavelength and second wavelength are measured. However, a biochip is generally formed to be thin in order to increase the portability and reduce the manufacturing cost, and thus the ratio of red blood cells may vary depending on the area. That is, when the volume ratios of the red blood cells in the first area (A1) and the second area (A2) are different from each other, the error rate would increase, which is a problem.

### SUMMARY

Various embodiments of the present disclosure are directed to an apparatus and method for measuring properties of a fluid that is capable of reducing measurement errors caused by the unhomogeneity of the fluid inside a biochip by emitting a plurality of lights having a plurality of wavelengths to a same area.

An embodiment of the present disclosure provides an apparatus for measuring properties of a fluid, the apparatus including: a light emitting unit configured to emit a first light having a first wavelength and a second light having a second wavelength that is longer than the first wavelength, from outside a fluid accommodating unit where the fluid flows in and out to a measurement area inside the fluid accommodating unit; a light receiving unit disposed outside the fluid accommodating unit and configured to receive the first light and second light that passed the measurement area; and a measuring unit configured to measure the properties of the fluid based on an intensity of the first light and second light that the light emitting unit emitted and an intensity of the first light and second light that the light receiving unit received.

Another embodiment of the present disclosure provides a method for measuring properties of a fluid, the method including: accommodating the fluid in a fluid accommodating unit to which the fluid may flow in and out; emitting, by a light emitting unit disposed outside the fluid accommodating unit, a first light having a first wavelength to a measurement area in the fluid accommodating unit; receiving, by a light receiving unit disposed outside the fluid accommodating unit, the first light that passed the measurement area; emitting, by the light emitting unit, a second light having a second wavelength that is longer than the first wavelength to the measurement area; receiving, by the light receiving unit, the second light that passed the measurement area; and measuring the properties of the fluid based on an intensity of the first light and second light that the light emitting unit emitted and an intensity of the first light and second light that the light receiving unit received.

Various aforementioned embodiments of the present disclosure have an effect of providing an apparatus and method for measuring properties of a fluid that is capable of reducing measurement errors caused by the unhomogeneity of the fluid inside a biochip by emitting a plurality of lights having a plurality of wavelengths to a same area.

### BRIEF DESCRIPTION OF THE DRAWINGS

Example embodiments will now be described more fully hereinafter with reference to the accompanying drawings; however, they may be embodied in is different forms and should not be construed as limited to the embodiments set forth herein. Rather, these embodiments are provided so that this disclosure will be thorough and complete, and will fully convey the scope of the example embodiments to those skilled in the art.

In the drawing figures, dimensions may be exaggerated for clarity of illustration. It will be understood that when an element is referred to as being “between” two elements, it can be the only element between the two elements, or one or more intervening elements may also be present. Like reference numerals refer to like elements throughout.

FIG. 1 is a view for explaining problems of a conventional apparatus for measuring properties of a fluid;

FIG. 2 is a view for explaining a concept of an apparatus for measuring properties of a fluid according to an embodiment of the present disclosure;

FIG. 3 is a view for explaining a light focusing unit of the apparatus for measuring properties of a fluid according to the embodiment of the present disclosure;

FIG. 4 is a view for explaining a concept of an apparatus for measuring properties of a fluid according to another embodiment of the present disclosure;

FIG. 5 is a view for explaining a concept of a light receiving unit of the apparatus for measuring properties of a fluid according to the another embodiment of the present disclosure;

FIG. 6 is a view for explaining a concept of a light receiving unit of the apparatus for measuring properties of a fluid according to the another embodiment of the present disclosure;

FIG. 7 is a flowchart for explaining a method for measuring properties of a fluid according to an embodiment of the present disclosure; and

FIGS. 8 and 9 are flowcharts for explaining emitting a light in the method for measuring properties of a fluid according to the embodiment of the present disclosure.

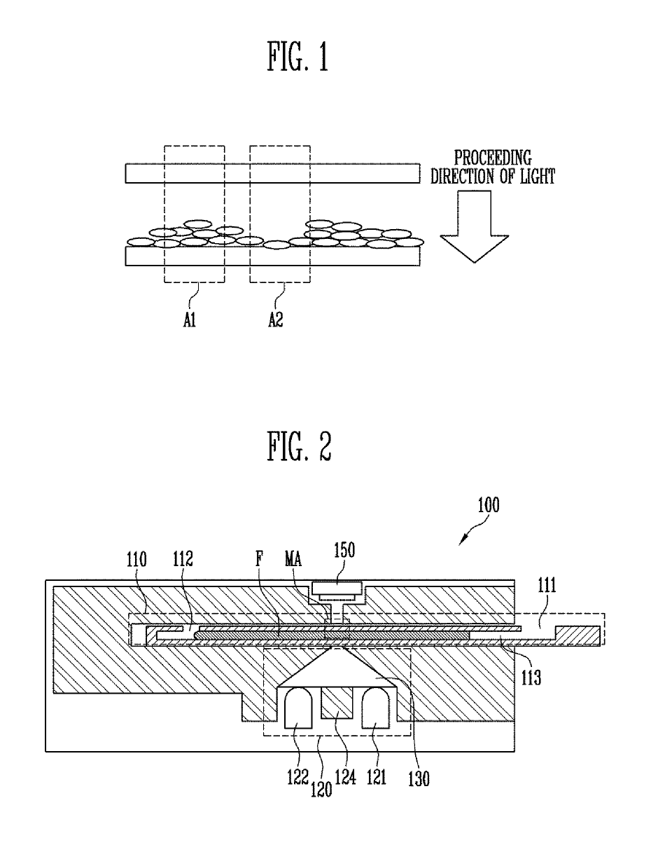

### DETAILED DESCRIPTION

Hereinafter, embodiments will be described in greater detail with reference to the accompanying drawings. Embodiments are described herein with reference to cross-sectional illustrations that are schematic illustrations of embodiments (and intermediate structures). As such, variations from the shapes of the illustrations as a result, for example, of manufacturing techniques and/or tolerances, are to be expected. Thus, embodiments should not be construed as limited to the particular shapes of regions illustrated herein but may include deviations in shapes that result, for example, from manufacturing. In the drawings, lengths and sizes of layers and regions may be exaggerated for clarity. Like reference numerals in the drawings denote like elements.

Terms such as ‘first’ and ‘second’ may be used to describe various components, but they should not limit the various components. Those terms are only used for the purpose of differentiating a component from other components. For example, a first component may be referred to as a second component, and a second component may be referred to as a first component and so forth without departing from the spirit and scope of the present disclosure. Furthermore, ‘and/or’ may include any one of or a combination of the components mentioned.

Furthermore, a singular form may include a plural from as long as it is not specifically mentioned in a sentence. Furthermore, “include/comprise” or “including/comprising” used in the specification represents that one or more components, steps, operations, and elements exist or are added.

Furthermore, unless defined otherwise, all the terms used in this specification including technical and scientific terms have the same meanings as would be generally understood by those skilled in the related art. The terms defined in generally used dictionaries should be construed as having the same meanings as would be construed in the context of the related art, and unless clearly defined otherwise in this specification, should not be construed as having idealistic or overly formal meanings.

It is also noted that in this specification, “connected/coupled” refers to one component not only directly coupling another component but also indirectly coupling another component through an intermediate component. On the other hand, “directly connected/directly coupled” refers to one component directly coupling another component without an intermediate component.

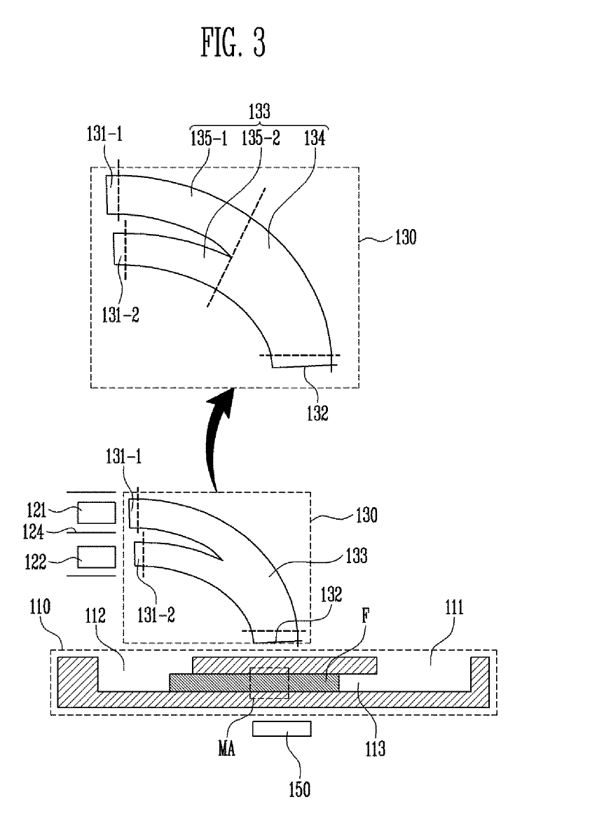

FIG. 2 is a view for explaining a concept of an apparatus for measuring properties of a fluid according to an embodiment of the present disclosure. The measuring apparatus 100 includes a light emitting unit 120, light receiving unit 150 and measuring unit (not illustrated), and when there is a fluid accommodating unit (biochip) 110 inserted into the measuring apparatus 100, the measuring apparatus 100 may measure properties of the fluid (F) accommodated in the fluid accommodating unit 110. The fluid accommodating unit 110 includes an inlet 111, outlet 112, and a passage 113 that connects the inlet 111 and outlet 112, and the fluid accommodating unit 110 may accommodate the fluid (F). In order to prevent vortex from occurring that interrupts flow of the fluid, it is desirable that a laminar flow is formed in the fluid flowing through the passage 113. A thickness of the passage 113 may desirably be 1 to 500 μm. Fabricating the passage 113 to have a thickness of 1 μm is very difficult due to fabricating errors, and the fluid may not flow smoothly. Furthermore, when fabricating the passage 113 to have a thickness of above 500 μm, vortex may be generated in the fluid, and measurement errors may increase, significantly reducing the reliability of the measurement. Furthermore, a thickness of the fluid accommodating unit 110 may desirably be 1 to 10 mm. When the thickness of the fluid accommodating unit 110 is less than 1 mm, areas where passages are formed may be damaged by impact, and when the thickness of the fluid accommodating unit 110 is less than 10 mm, the price may increase and the portability may decrease. For optical measurement, the fluid accommodating unit 110 may desirably be made of a transparent material. The emitting unit 120 emits a first light having a first wavelength and a second light having a second wavelength that is longer than the first wavelength to a measurement area (MA) in the fluid accommodating unit 110. The light receiving unit 150 receives the first light and second light that passed the measurement area (MA), and the measuring unit (not illustrated) measures properties of the fluid based on an intensity of the first light and second light that the light emitting unit 120 emitted and an intensity of the first light and second light that the light receiving unit 150 received.

The light emitting unit 120 includes a first light generating unit 121 that generates the first light, a second light generating unit 122, a light shield wall 124 and a light focusing unit 130. The first light generating unit 121 generates the first light, and the second light generating unit 122 generates the second light and is adjacent to the first light generating unit 121. The first light and second light are emitted alternately, and the light shield wall 124 prevents the first light and second light from being mixed together. The light focusing unit 130 focuses the first light generated by the first light generating unit 121 and the second light generated by the second light generating unit 122 to be emitted to a same measurement area (MA). Details of such a structure will be explained hereinafter.

The light receiving unit 150 may receive the first light and second light that passed the measurement area (MA), and the light receiving unit 150 may include a photo diode, CIS, or CCD.

The measuring unit (not illustrated) stores a math equation and correcting constant, and measures properties of the fluid (F) based on an intensity of the first light and second light that the light emitting unit 120 emitted and an intensity of the first light and second light that the light receiving unit 150 received. In a case where the fluid (F) is blood, it is possible to measure a volume ratio of red blood cells to an entirety of the blood by optical measurement. Since the first light and second light are emitted alternately, the measuring unit (not illustrated) may determine whether or not the received light is the first light or second light based on a time when the light is received by the receiving unit 150.

An electromagnetic transmission rate (A) of the first light and second light may be calculated through the math equation shown below.

$$T = \frac{{I}_{1}}{{I}_{0}} = {e}^{- α lc} = {e}^{- A} \qquad 〈 Math equation 1 〉$$

Herein, T represents the transmission rate, I₁ represents an intensity of the light (first light or second light) after it has been transmitted through the fluid is accommodating unit 110, I₀ represents an intensity of the light before it is transmitted through the fluid accommodating unit 110, α represents a damping constant per mol, 1 represents a transmission passage, c represents a concentration, and A represents an electromagnetic absorption ratio. In a hematocrit measurement, light having a wavelength of 570 nm or light having a wavelength of 880 nm may be used. After obtaining the electromagnetic absorption ratio of each light, a hematocrit may be calculated through the math equation shown below.

$$HCT = \frac{{c}^{570} {A}^{570}}{{c}^{570} {A}^{570} + {c}^{880} {A}^{880}} \qquad 〈 Math equation 2 〉$$

Herein, HCT is a volume ratio of red blood cells to an whole blood, A₅₇₀ and A₈₈₀ are light absorption ratios at 570 nm and 880 nm, respectively, c₅₇₀ and c₈₈₀ are correcting constants at 570 nm and 880 nm, respectively. That is, the measuring unit (not illustrated) stores math equation 1, math equation 2, c₅₇₀ and C₈₈₀.

FIG. 3 is a view for explaining a light focusing unit of the apparatus for measuring properties of a fluid according to the embodiment of the present disclosure. Referring to FIG. 3, the light focusing unit 130 includes a light focusing inlet 131-1, 131-2, light focusing outlet 132, and light focusing passage 133.

The light focusing inlet 131-1, 131-2 includes a first light focusing inlet 131-1 where the first light is emitted and a second light focusing inlet 131-2 where the second light is emitted, and the light focusing outlet 132 transmits the first light and second light to the measurement area (MA).

The light focusing passage 133 connects the light focusing inlet 131-1, 131-2 to the light focusing outlet 132, and the light focusing passage 133 includes a light stem unit 134 of which one end is connected to the light focusing outlet 132, a first light branch unit 135-1 of which one end is connected to the first light focusing inlet 131-1 and another end connected to a portion of another end of the light stem unit 134, and a second light branch unit 135-2 of which one end is connected to the second light focusing inlet 131-2 and another end connected to at least a portion of the another end of the light stem unit 134 not connected to the first light branch unit 135-1. A portion of the surface of the light focusing passage 133 that is connected to the light focusing inlet 131-1, 131-2 and light focusing outlet 132 may transmit light, but at least a portion of the rest of the surface maximizes the amount of the first light and second light arriving at the light receiving unit 150 by reflecting the first light and second light. For example, at least one selected from glass, PMMA (polymethyl methacrylate), PI (Polyimide), PC (Polycarbonate) and COC (cyclo olefin copolymer) may constitute the light focusing passage 133, and in a case where the surface of the light focusing passage 133 is a curved surface that is not bent, the light focusing passage 133 may reflect the first light and second light due to the difference of refractive index of air and the light focusing passage 133. Alternatively, at least one selected from Au, Ag and Al may constitute the surface of the light focusing passage 133, and due to optical characteristics of the surface of the light focusing passage 133, the light focusing passage 133 may reflect the first light and second light. At least one selected from glass PMMA (polymethyl methacrylate), PI (Polyimide), PC (Polycarbonate) and COC (cyclo olefin copolymer) may constitute the rest of the light focusing passage 133 besides the surface thereof.

The first light generated by the first light generating unit 121 is emitted to the first light focusing inlet 131-1, passes the first light branch unit 135-1 and light stem unit 134, and arrives at the light focusing outlet 132. The second light generated by the second light generating unit 122 is emitted to the second light focusing inlet 131-2, passes the second light branch unit 135-2 and light stem unit 134, and arrives at the light focusing outlet 132. Therefore, the light focusing unit 134 focuses the first light and second light generated in different areas and emits the focused light to the measurement area (MA).

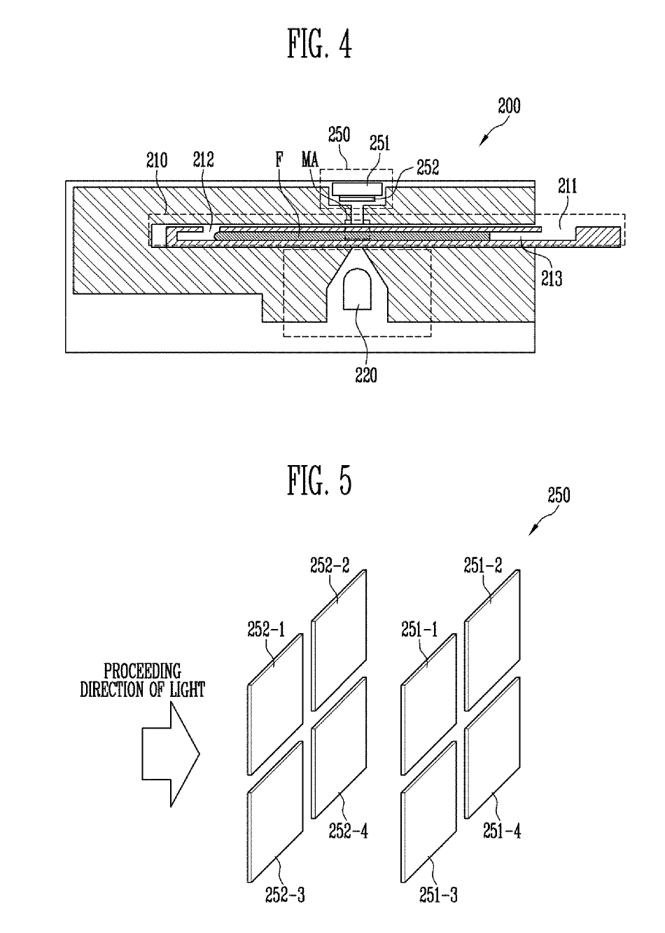

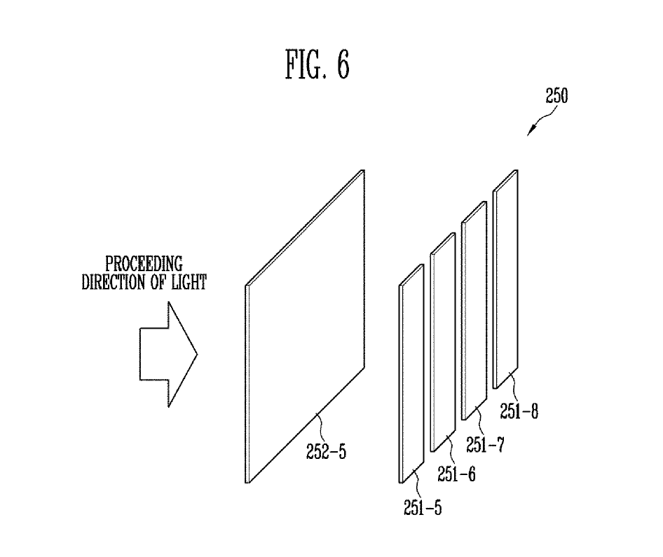

FIG. 4 is a view for explaining a concept of an apparatus for measuring properties of a fluid according to another embodiment of the present disclosure; FIG. 5 is a view for explaining a concept of a light receiving unit of the apparatus for measuring properties of a fluid according to the another embodiment of the present disclosure; and FIG. 6 is a view for explaining a concept of a light receiving unit of the apparatus for measuring properties of a fluid according to the another embodiment of the present disclosure. Hereinafter, explanation will be made with reference to FIGS. 4 to 6.

A measuring apparatus 200 includes a light emitting unit 220, light receiving unit 250, and measuring unit (not illustrated). In a case where there is a fluid accommodating unit 210 inserted in the measuring apparatus 200, the measuring apparatus 200 may measure properties of a fluid (F) accommodated in the fluid accommodating unit 210. The fluid accommodating unit 210 is the same as the fluid accommodating unit 110 of FIG. 2, and thus detailed explanation will be omitted. The light emitting unit 220 includes a broadband light source that emits a broadband light that includes both a first light and second light. The broadband light may be transmitted through the measurement area (MA) and arrive at the light receiving unit 250.

The light receiving unit 250 includes a plurality of light receiving areas 251 that includes a first light receiving area 251-1, second light receiving area 251-2, third light receiving area 251-3, and fourth light receiving area 251-4, and a light division unit 252. The light division unit 252 receives the broadband light, and transmits a light having a different wavelength to each of the light receiving areas 251-1, 251-2, 251-3, and 251-4. Each of the light receiving areas 251-1, 251-2, 251-3, and 251-4 may include a photodiode, CIS, or CCD.

The measuring unit (not illustrated) is very similar to the measuring unit (not illustrated) explained with reference to FIG. 2, and thus detailed explanation will be omitted. In FIG. 2, the first light and second light are emitted alternately, and thus a wavelength of the light received is determined by the measuring unit based on a time when the light is received in the light receiving unit 150. However, in FIG. 5, the light receiving areas 251-1, 251-2, 251-3, and 251-4 receive lights of different wavelengths, and thus the measuring unit (not illustrated) may determine the wavelength of the light that each light receiving areas 251-1, 251-2, 251-3, and 251-4 receives based on an index 1, 2, 3, and 4 of each of the light receiving areas 251-1, 251-2, 251-3, and 251-4.

Referring to FIG. 5, the light division unit 252 includes a plurality of filters 252-1, 252-2, 252-3, and 252-4 corresponding to the plurality of light receiving areas 251-1, 251-2, 251-3, and 251-4. Each of the plurality of filters 252-1, 252-2, 252-3, 252-4 transmits only a certain wavelength and delivers it to each of the plurality of light receiving areas 251-1, 251-2, 251-3, and 251-4. The first filter 252-1 transmits a light having a first wavelength to the first light receiving area 251-1, the second filter 252-2 transmits a light having a second wavelength to the second light receiving area 251-2, the third filer transmits a light having a third wavelength to the third light receiving area 251-3, and the fourth filter 252-4 transmits a light having the fourth wavelength to the fourth light receiving area 251-4. Herein, the first wavelength, second wavelength, third wavelength and fourth wavelength are all different from one another.

Referring to FIG. 6, the light division unit 252-5 includes a fine structure unit (not illustrated). The fine structure unit (not illustrated) may transmit only a plurality of certain wavelengths. Furthermore, in a case where a size and material of the fine structure unit (not illustrated) may be adequately adjusted, a light emitted to the light division unit 252-5 may be divided to have a different passage depending on its wavelength. Accordingly, a light having a fifth wavelength, sixth wavelength, seventh wavelength, or eighth wavelength that are different from one another may be transmitted to each of the light receiving areas 251-5, 251-6, 251-7, and 251-8.

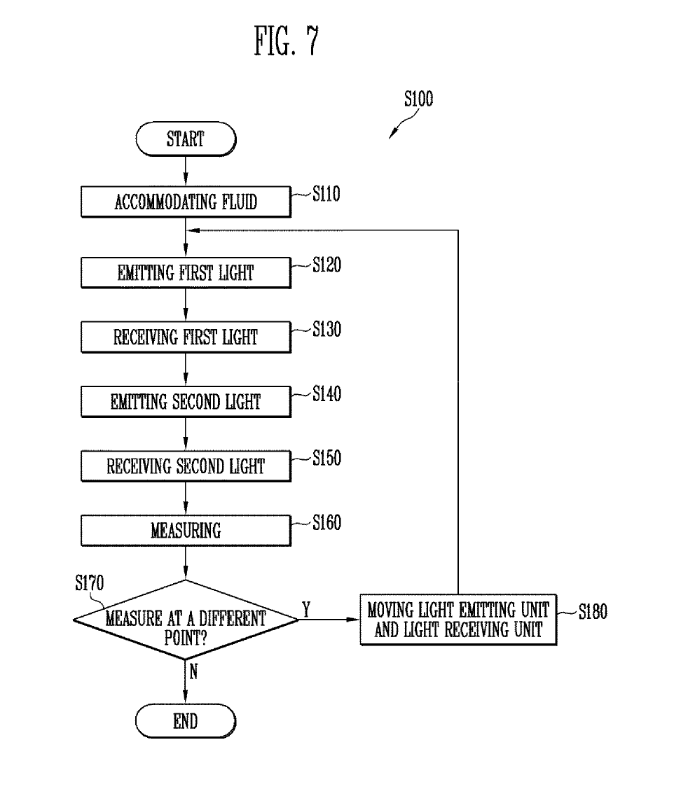

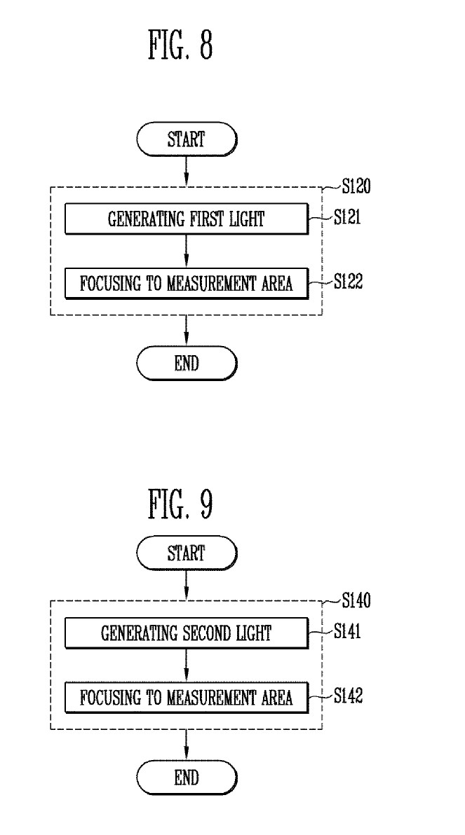

FIG. 7 is a flowchart for explaining a method for measuring properties of a fluid according to another embodiment of the present disclosure, and FIGS. 8 and 9 are flowcharts for explaining emitting light of the method for measuring properties of a fluid according to the another embodiment of the present disclosure. Hereinafter, explanation will be made with reference to FIGS. 2, 3, 7, 8, and 9.

Referring to FIG. 7, a method for measuring properties of a fluid according to an embodiment of the present disclosure includes accommodating the fluid (S110), emitting the first light (S120), receiving the first light (S130), emitting the second light (S140), receiving the second light (S150), measuring (S160), (S170), and moving the light emitting unit and light receiving unit (S180).

At the step of accommodating the fluid (S110), the fluid (F) is accommodated in the fluid accommodating unit 110 that includes the inlet 111, outlet 112, and the passage 113 connecting the inlet 111 and outlet 112. Furthermore, the fluid accommodating unit 110 is inserted in the measuring apparatus 100.

At the step of emitting the first light (S120), the first light generating unit 121 generates the first light having the first wavelength (S121). Then, the first light is focused as it passes the first light focusing inlet 131-1, first light branch unit 135-1, light stem unit 134 and light focusing outlet 132 (S122), and then emitted to the measurement area (MA) inside the fluid accommodating unit 110.

At the step of receiving the first light (S130), the light receiving unit 150 receives the first light that passed the measurement area (MA). Since the time the light receiving unit 150 received light corresponds to the time when the first light generating unit 121 generated the first light, the measuring unit (not illustrated) determines that the light received in the light receiving unit 150 is the first light.

At the step of emitting the second light (S140), the second light generating unit 122 generates the second light having the second wavelength that is longer than the first wavelength (S141). Then, the second light is focused as it passes the second light focusing inlet 131-2, second light branch unit 135-2, light stem unit 134 and light focusing outlet 132 (S142), then emitted to the measurement area (MA) inside the fluid accommodating unit 110.

At the step of receiving the second light (S150), the light receiving unit 150 receives the second light that passed the measurement area (MA). In the same manner as in the step of receiving the first light, the measuring unit (not illustrated) determines that the light received in the light receiving unit 150 is the second light.

At the step of measuring (S160), the measuring unit (not illustrated) stores the math equation and correcting constant, and measures properties of the fluid (F) based on the intensity of the first light and second light that the light emitting unit 120 emitted and the intensity of the first light and second light that the light receiving unit 150 received. The math equation and correcting constant stored in the measuring unit (not illustrated) and the method of measuring the properties of the fluid were explained hereinabove.

At the step (S170), in a case where it is necessary to move the measurement area (MA) for the same fluid (F) and perform an additional measurement, the step of moving the light emitting unit and light receiving unit is performed (S180), and in a case where it is not necessary to move the measurement area (MA) nor perform an additional measurement, the method for measuring the properties of the fluid (S100) ends. Before or during performing the method for measuring the properties of the fluid (S100), the position and number of the measurement area (MA) may be input by the user.

At the step of moving the light emitting unit and light receiving unit (S180), the light emitting unit 120 and light receiving unit 150 are moved so that the input measurement area (MA) may be measured. After the step of moving the light emitting unit and light receiving unit (S180), the step of emitting the first light for measurement (S120) is performed.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. An apparatus for measuring properties of a fluid, the apparatus comprising:
a light emitting unit configured to emit a first light having a first wavelength and a second light having a second wavelength that is longer than the first wavelength, from outside a fluid accommodating unit where the fluid flows in and out to a measurement area inside the fluid accommodating unit;
a light receiving unit disposed outside the fluid accommodating unit and configured to receive the first light and second light that passed the measurement area; and
a measuring unit configured to measure the properties of the fluid based on an intensity of the first light and second light that the light emitting unit emitted and an intensity of the first light and second light that the light receiving unit received.

2. The apparatus according to claim 1,
wherein the light emitting unit comprises:
a first light generating unit configured to generate the first light;
a second light generating unit adjacent to the first light generating unit and configured to generate the second light; and
a light focusing unit configured to focus the first light and second light so that the first light and second light may be emitted to the measurement area.

3. The apparatus according to claim 2,
wherein the first light and second light are emitted alternately, and the measuring unit determines whether the light is the first light or the second light based on a time when the light receiving unit received the light.

4. The apparatus according to claim 2,
wherein the light focusing unit comprises:
a light focusing inlet to which the first light and second light are emitted;
a light focusing outlet configured to transmit the first light and second light emitted to the light focusing inlet to the measurement area; and
a light focusing passage connecting the light focusing inlet and light focusing outlet.

5. The apparatus according to claim 4,
wherein at least a portion of a surface of the light focusing passage reflects the first light and second light.

6. The apparatus according to claim 5,
wherein at least one selected from Au, Ag and Al constitutes the surface of the light focusing passage, and the light focusing passage reflects the first light and second light due to optical characteristics of the light focusing passage.

7. The apparatus according to claim 5,
wherein at least one selected from glass, PMMA (polymethyl methacrylate), PI (Polyimide), PC (Polycarbonate) and COC (cyclo olefin copolymer) constitutes the light focusing passage, and the light focusing passage reflects the first light and second light due to a difference of refractive index between air and the light focusing passage.

8. The apparatus according to claim 4,
wherein the light focusing inlet comprises:
a first light focusing inlet to which the first light is emitted; and
a second light focusing inlet adjacent to the first light focusing inlet and to which the second light is emitted, and
the light focusing passage comprises:
a light stem unit of which one end is connected to the light focusing outlet;
a first light branch unit of which one end is connected to the first light focusing inlet and of which another end is connected to a portion of another end of the light stem unit; and
a second branch unit of which one end is connected to the second light focusing inlet and of which another end is connected to at least a portion of the another end of the light stem unit that is not connected to the first light branch unit.

9. The apparatus according to claim 1,
wherein the light emitting unit comprises a broadband light source that emits a broadband light that includes the first light and second light,
the light receiving unit comprises a plurality of light receiving areas, and a light division unit configured to receive the broadband light from the broadband light source and transmit a light having a different wavelength to each of the light receiving areas,
the measuring unit determines wavelength of the light received by each light receiving area based on index of the light receiving area.

10. The apparatus according to claim 9,
wherein the light division unit comprises a plurality of filters corresponding to the plurality of light receiving areas, each filter transmitting the light having a different wavelength and delivering it to each of the light receiving areas.

11. The apparatus according to claim 9,
wherein the light division unit comprises a fine structure unit configured to change a light passage depending on a wavelength and to deliver the light having a different wavelength to each of the light receiving areas.

12. The apparatus according to claim 1,
wherein the fluid accommodating unit accommodates an whole blood, and
the measuring unit measures a volume ratio of red blood cells to the whole blood.

13. The apparatus according to claim 1,
wherein the fluid flowing through the passage forms a laminar flow, and a height of the passage is 1 to 500 μm.

14. A method for measuring properties of a fluid, the method comprising:
accommodating the fluid in a fluid accommodating unit to which the fluid may flow in and out;
emitting, by a light emitting unit disposed outside the fluid accommodating unit, a first light having a first wavelength to a measurement area in the fluid accommodating unit;
receiving, by a light receiving unit disposed outside the fluid accommodating unit, the first light that passed the measurement area;
emitting, by the light emitting unit, a second light having a second wavelength that is longer than the first wavelength to the measurement area;
receiving, by the light receiving unit, the second light that passed the measurement area; and
measuring the properties of the fluid based on an intensity of the first light and second light that the light emitting unit emitted and an intensity of the first light and second light that the light receiving unit received.

15. The method according to claim 14,
wherein the emitting the first light comprises:
generating the first light; and focusing the first light to the measurement area, and
the emitting the second light comprises:
generating the second light; and focusing the second light to the measurement area.

16. The method according to claim 14,
further comprising moving the light emitting unit and light receiving unit after the measuring of the properties of the fluid.

17. The method according to claim 14,
wherein, at the accommodating of the fluid, the fluid is an whole blood, and
the measuring of the properties of the fluid involves measuring a volume ratio of red blood cells to the whole blood.
